(19) United States
(12) Patent Application Publication
(10) Pub. No.: US 2025/0221725 A1
(43) Pub. Date: Jul. 10, 2025

(54) MEDICAL TOOL TIP, ACTUATION DEVICE, SURGICAL INSTRUMENT, AND SECURING METHOD

(71) Applicant: KAMMERER MEDICAL SYSTEMS GMBH & CO. KG, Stockach (DE)

(72) Inventors: Helena SCHREMPP, Stockach (DE); Rüdiger SCHWÄGLER, Stockach (DE)

(21) Appl. No.: 18/852,927

(22) PCT Filed: Mar. 30, 2023
(86) PCT No.: PCT/DE2023/100247

§ 371 Date: Sep. 30, 2024

(30) Foreign Application Priority Data
Mar. 31, 2022 (DE) 10 2022 107 751.9

(51) Int. Cl. A61B 17/29; A61B 17/00

(52) U.S. Cl. CPC A61B 17/2909; A61B 2017/00477; A61B 2017/2925; A61B 2017/2931; A61B 2017/294

(57) ABSTRACT

A medical tool tip has a first tip part with a first securing section for securing to a main part of the actuation device without tools and a second tip part with a second securing section for securing to a slide of the actuation device without tools. The second tip part is secured to the first tip part in a longitudinally movable manner at least in some sections. The first and second securing section are designed such that the second tip part can be secured to the slide only when the first securing section is already secured to the main part.

### FIELD

The present disclosure relates to a medical tool tip, an actuation device, a surgical instrument, and a method for securing a medical tool tip to an actuation device.

### INTRODUCTION

In some medical interventions, surgical instruments are used to remove or extract bones, cartilage or other tissue. Such instruments usually have a longitudinal shank on which a medical tool is provided at the distal end, i.e. the end remote from the user. The user can actuate the tool, e.g. a punch or spoon forceps, via a mechanism at the proximal end of the shank, i.e. the end nearest the user.

In practice, it frequently occurs that during an intervention, different tools are required and/or a tool is required in multiple sizes. In view of procurement costs, storage capacity and practicability, therefore surgical instruments have been developed which consist of an actuation device and an interchangeable medical tool tip. Depending on requirements therefore, the desired tool tips can be attached successively to a single actuation device, which is held by the user and the proximal end of which comprises the mechanism for actuating the tool. This may also improve safety during interventions by immediate replacement if the tool tip becomes bent or blunt, and improve durability since it is not necessary to dispose of the whole instrument, but only the tip.

### SUMMARY

In this context, it is an object of the present disclosure to further improve the user-friendliness during use of a surgical instrument or at least parts thereof.

This object is achieved by a medical tool tip, an actuation device, a surgical instrument and a method for securing a medical tool tip to an actuation device, as described below.

A first aspect of the present disclosure relates to a medical tool tip for a surgical instrument. The tool tip has a first, preferably one-piece tip part with a first attachment portion for toolless attachment of the first tip part to a base body of an actuation device, and a second, preferably one-piece or two-piece tip part with a second attachment portion for toolless attachment of the second tip part to a slide of the actuation device. The second tip part is at least partially longitudinally displaceably, in particular toollessly inseparably, attached to the first tip part. According to the present disclosure, the first and second attachment portions are configured, in particular arranged relative to one another or able to be arranged by longitudinal displacement, such that the second tip part can be attached to the slide, in particular by at least partial insertion of the second attachment portion into the slide, only when the first tip part has already been attached, in particular by form fit, to the base body and thereby secured.

An aspect of the present disclosure is based on the approach of allowing creation of a connection between a medical tool tip, with a first tip part and a second tip part attached longitudinally displaceably thereon, and an actuation device by conventional actuation of the actuation device. For this, it is firstly suitable that (i) a slide of the actuation device can be brought, by a longitudinal displacement relative to a base body of the actuation device, into an attachment position in which the first tip part can be attached to the base body, and (ii) the slide—with the first tip part attached—can be connected to the second tip part by at least partial longitudinal displacement out of the attachment position. Secondly, for this it is also suitable that a first attachment portion of the first tip part for attachment to the base body, and a second attachment portion of the second tip part for attachment to the slide, are configured, in particular arranged or able to be arranged, e.g. spaced from one another in a longitudinal direction of the tool tip, such that the second tip part can be attached to the slide in particular also after the first attachment portion has already been attached to the base body. To this extent, it is preferred that the first and second attachment portions are configured, in particular arranged e.g. spaced, such that the tool tip can be attached to the actuation device in two stages.

The slide may for example be configured such that only in the attachment position does it allow insertion of the first attachment portion into a base body attachment portion for toolless, in particular form-fit, attachment to the base body. On attachment of the first tip part to the base body, the second attachment portion is suitably not yet connected to the slide, preferably because of its arrangement relative to the second attachment portion. By attachment to the base body, the first tip part can however be preferably fixed so that a slide attachment portion pushes over the second attachment portion during a longitudinal displacement of the slide—as suitably triggered by an actuation of the actuation device—and thus a toolless, in particular form-fit connection is also created between the slide and the second tip part.

An attachment device configured thus firstly, and/or a tool tip configured thus secondly, not only allows a toolless attachment of the tool tip to the actuation device but also an attachment with particularly few manual actions. A conventional actuation of the actuation device for a longitudinal displacement of the slide out of the attachment position, for connection to the second tip part, is thus in principle possible with one hand. In other words, the actuation device may be held with one hand and also actuated by this hand, and thereby the tool tip completely attached to the actuation device. To this extent, it is not necessary to actuate a separate locking mechanism. Without such a separate locking mechanism, there is also no risk of accidental triggering during use. Thus different tool tips can be rapidly and safely changed, even under stress and in sterile conditions, such as for example directly in an operating theatre.

Preferred embodiments of the present disclosure and their refinements are described below, each of which—unless expressly excluded—may be combined arbitrarily with one another and with the aspects of the present disclosure described below.

In a preferred embodiment, the first attachment portion and the second attachment portion have at least partially substantially the same contour. In other words, the first tip part in the first attachment portion and the second tip part in the second attachment portion are preferably configured such that, at least partially and from a predefined viewing angle, they have the same outline. The first and second attachment portions need not necessarily have the same dimensions. In particular, it is conceivable that the first attachment portion is formed at least partially smaller, in particular narrower, than the second attachment portion. This allows simple and intuitive attachment of the first attachment portion to the base body, since in this way the slide attachment portion, which is actually configured for connection of the second tip part, can serve to guide the first attachment portion into the base body attachment portion.

To this extent, it is preferred that the second attachment portion protrudes laterally on both sides over the first attachment portion transversely to a longitudinal extent of the tool tip. In other words, the first tip part in the first attachment portion is formed narrower than the second tip part in the second attachment portion. This may ensure that only the first tip part can be attached to the base body, and not the second tip part. In particular, it may thus be prevented that the punch tips are attached to the actuation device incorrectly, in particular the wrong way round, i.e. the second tip part attached to the base body.

As an alternative to the different dimensioning, in particular width, of the first and second attachment portions, other means for preventing incorrect attachment are also conceivable. For example, on a side opposite the second tip part, the first tip part may have a groove for receiving a guide rail of the base body. Thus the first and second attachment portions can only be completely received by the actuation device when the groove faces the guide rail. To this extent, in some embodiments, the tool tips may quite generally be configured to prevent incorrect attachment to the actuation device.

Preferably, the first and second attachment portions have the same contour in a contour plane. For example, the first tip part may have a top side which faces the second tip part with a flat first contact face. Accordingly, the second tip part suitably has an underside facing the first tip part with a flat second contact face. The second contact face of the second tip part may contact the first contact face in at least one operating position. Preferably, the first and second contact faces lie in the contact plane at least during this contact. The first contact face and the second contact face are preferably at least partially similarly formed. The term “similarly formed” may here in particular mean a form of two contact faces in which the contact faces have the same shape. The same dimensions, i.e. the same sizes, are not absolutely necessary. In particular, the first contact face may be smaller than the second contact face. The matching contours of the two attachment portions can be created by means of contact faces formed in this way.

In a further preferred embodiment, the first attachment portion has a first protrusion which, running longitudinally, adjoins a proximal end of the first tip part. Alternatively or additionally, it is preferred that the second attachment portion has a second protrusion which, running longitudinally, adjoins a proximal end of the second tip part. A particularly robust connection of the tool tip to the actuation device is possible by means of such protrusions. In particular, this may prevent the tool tip from falling out of the actuation device during use when the latter is rotated by 180° about its longitudinal axis. Also, the tool tip may thus be connected to the actuation device without formation of a step.

The first protrusion suitably has a top side with a flat top side face. Alternatively or additionally, the second protrusion has an underside with a flat underside face. Preferably, the underside face is oriented substantially parallel to the top side face. With such a design of the first and second protrusions, a smooth actuation of the tool tip can be ensured.

A particularly secure and form-fit attachment of the tool tip to the actuation device can be achieved if the first and second protrusions preferably have respective side walls, which run substantially perpendicularly to the top side face and underside face and are formed similarly uneven.

For example, the first tip part in the first attachment portion may have a top side with a top side face and a first bulge, which runs transversely to a surface normal of the top side face. Alternatively or additionally, the second tip part in the second attachment portion may have an underside with an underside face facing the top side face and a second bulge, which runs transversely to a surface normal of the underside face and is formed similarly to the first bulge. Here too, the term “formed similarly” means in particular that the forms have the same shape, e.g. the same contour. The bulges need not however necessarily be made the same size. The bulges allow a form-fit attachment to the actuation device, in particular an undercut by the base body attachment portion or slide attachment portion. By means of the similar bulges, the tool tips can also be reliably removed from the actuation device. If these are arranged substantially aligned with one another, it is possible to conduct the first attachment portion out through a recess in the slide, in which the second attachment portion is arranged during operation.

Suitably, the first tip part and/or the second tip part each have a convex curvature in the region of the respective first or second bulge. Thus in particular, in the slide attachment portion, the slide may be bent when pushed onto the second tip part, so that a part of the slide attachment portion engages behind the second bulge.

In a further preferred embodiment, the tool tip is configured as a sterile single-use tool. This may substantially simplify the logistics, e.g. in a hospital, since the tool tip need not be sterilized after use. Also, in this way new and hence sharp tool tips can always be provided. Because of the guaranteed sterility of such a single-use tool, patient safety may also be increased.

Alternatively however, it is also possible that the tool tip is formed to be reusable, i.e. in particular cleanable and/or sterilizable. This may in some cases reduce the complexity of procurement and/or costs.

A second aspect of the present disclosure relates to an actuation device for a surgical instrument. The actuation device has (i) a base body with a base body attachment portion for toolless, in particular form-fit, attachment of a first tip part of a medical tool tip to the base body, and (ii) a slide with a slide attachment portion for toolless, in particular form-fit, attachment of a second tip part of the tool tip, which is longitudinally displaceably, in particular toollessly inseparably, attached to the first tip part, to the slide. Here, the slide is at least partially longitudinally displaceably, in particular toollessly inseparably, attached to the base body. According to the present disclosure, for toolless attachment of the tool tip to the actuation device, the slide (i) can be brought into an attachment position by at least partial longitudinal displacement relative to the base body, in which position the first tip part can be attached to the base body in the base body attachment portion.

The slide is also (ii) configured such that, when the first tip part is attached to the base body, the second tip part can be connected, in particular at least partially inserted in the slide attachment portion, to the slide in the slide attachment portion by at least partial longitudinal displacement of the slide out of the attachment position.

The actuation device may be configured such that the slide is raised from the base body in the attachment position. In other words, a gap may form between the slide and the base body on longitudinal displacement of the slide into the attachment position. The slide and base body are here suitably oriented substantially parallel to one another, as with conventional actuation of the actuation device.

Such an actuation device is particularly user-friendly, since thus for example a rapid change of different tool tips is possible even under stress and in sterile conditions, such as directly in the operating theatre. This may, inter alia, lead to shorter operation times. Also, by means of the actuation device, the space required on an instrument tray may be reduced, since there is no longer a need for multiple complete surgical instruments for different applications.

In a preferred embodiment, a contour of the base body attachment portion and a contour of the slide attachment portion at least partially substantially align with one another when the slide is in the attachment position. This allows for example the first attachment portion to be conducted through the slide attachment portion into the base body attachment portion for attachment of the first tip part to the base body.

In the sense of the present disclosure, two components or their contours here in particular at least partially substantially align with one another when the essential features of the components or contours, such as corners or edges or other significant protrusions, are oriented to one another. The term “align” thus does not mean absolute congruence. Rather, it may mean a relative position of two components in which the components or their contours, in particular their significant portions, correspond to one another. To this extent, two components may at least partially substantially align with one another if the one component is dimensioned slightly larger than the other component.

An at least partial substantial alignment of the contours of the base body attachment portion and slide attachment portion is possible for example if the base body has a base body top side facing the slide with a flat base body sliding face. Accordingly, the slide may have a slide underside facing the base body with a flat slide sliding face, via which the slide at least partially slides along the base body sliding face on a longitudinal displacement relative to the base body, e.g. until the slide lifts away from the base body. This may facilitate smooth operation of the actuation device.

In order not to hinder an attachment of the first tip part to the base body, it is preferred that the base body sliding face and the slide sliding face at least partially substantially align with one another when the base body and the slide are in the attachment position.

In particular, the base body attachment portion may have a base body recess, and the slide attachment portion may have a slide recess, which at least partially substantially align with one another in the attachment position. The base body recess and the slide recess suitably allow the in particular form-fit receiving of the first attachment portion or second attachment portion respectively. By the at least substantial alignment of these recesses in the attachment position of the slide, in particular the first attachment portion can be inserted through the slide attachment portion into the base body attachment portion. The slide recess may here serve as a guide. Conversely, on detachment of the tool tip from the actuation device, the alignment may allow in particular removal of the first attachment portion through the slide. To this extent, by the alignment, the two tip parts may be simultaneously detached from the actuation device.

In a further preferred embodiment, the contour of the slide attachment portion no longer aligns—in particular no longer completely and substantially—with the contour of the base body attachment portion when the slide is or becomes longitudinally displaced relative to the base body out of the attachment position. This allows the first tip part to be reliably secured on the base body.

To this extent, it is preferred that the slide attachment portion is configured to secure first tip part, which is attached to the base body attachment portion. The term “secure” here in particular may mean a fixing and/or locking of the first tip part, in particular of the first attachment portion. In other words, the slide attachment portion is suitably configured, in particular formed, such that after a longitudinal displacement out of the attachment position, it can prevent detachment of the first tip part from the base body. To this extent, the slide attachment portion may serve not only to create a connection between the slide and the second tip part, but also to attach the first tip part to the base body.

Accordingly, conversely it may also be provided that the slide is configured to release the first tip part, which is attached to the base body attachment portion, in the attachment position. In other words, the slide attachment portion may be configured such that the first tip part can only be detached from the base body when the slide is in the attachment position. Thus an accidental detachment of the tool tip from the actuation device may be countered.

In a further preferred embodiment, the slide in the slide attachment portion is formed as a fork for at least partially surrounding the second tip part. In particular, in the slide attachment portion, the slide may be configured for laterally surrounding the second tip part, in particular the second attachment portion. The term “laterally surrounding” here may mean a surrounding of (cross-sectional) faces, the surface normals of which are oriented parallel to surface normals of the first and second sliding faces. Because of the forked design of the slide, the second attachment portion can be inserted into the slide attachment portion, i.e. “frontally” into the slide, by a longitudinal displacement of the slide relative to the base body out of the attachment position.

For reliable, in particular lateral, surrounding of the second tip part in the second attachment portion, the slide attachment portion preferably has two fork tines. The fork tines may be configured as blades, i.e. thin with respect to a longitudinal extent. This may reduce the stiffness of the fork tines.

To this extent, it is particularly preferred that the fork tines are designed to be deformable for attachment of the second tip part by longitudinal displacement of the slide. In other words, for pushing onto the second tip part, the fork tines may be designed to be in particular laterally bendable. Thus the slide attachment portion may for example be pushed over—and undercut—an in particular lateral bulge of the second attachment portion.

To this extent, it is particularly preferred that the slide attachment portion is configured to engage on the second tip part for attachment. To this extent, the slide attachment portion together with the second attachment portion may form a latching mechanism, so that the slide attachment portion engages on the second attachment portion on the second tip part on actuation of the actuation device and the resulting longitudinal displacement of the slide. This allows simple and straightforward creation of a connection between the slide and the second tip part. At the same time, the engagement, which is triggered automatically by actuation of the actuation device, may increase the security against unintended detachment of the tool tip from the actuation device. Thus further separate securing systems, such as e.g. levers or latches, may be omitted.

A third aspect of the present disclosure concerns a surgical instrument. The instrument suitably has an actuation device according to the second aspect of the present disclosure and a medical tool tip according to the first aspect of the present disclosure. Such an instrument can be operated easily and safely, in particular because of the automatic securing and engagement during actuation. The reusability of at least the actuation device may also save raw materials, storage space and energy. To this extent, the instrument is particularly environmentally friendly and economic.

A fourth aspect of the present disclosure concerns a method for securing a medical tool tip, in particular according to the first aspect of the present disclosure, to an actuation device, in particular according to the second aspect of the present disclosure. The medical tool tip suitably has a first tip part and a second tip part which is attached longitudinally displaceably, in particular toollessly inseparably, on the first tip part. The actuation device suitably has a base body and a slide which is attached longitudinally displaceably, in particular toollessly inseparably, on the base body. In the method, the slide is brought into an attachment position by longitudinal displacement relative to the base body, and the first tip part is toollessly attached, in particular by form fit, by means of a first attachment portion, to the base body in a base body attachment portion.

Also, in particular following this, the second tip part is toollessly attached, in particular by form fit, by means of a second attachment portion, to the slide in a slide attachment portion by longitudinal displacement of the slide relative to the base body out of the attachment position.

This method allows a rapid exchange of tool tips even under stress and in sterile conditions, for example in the operating theatre. In particular, sharp cutting tool tips can be replaced more easily and quickly, which can lead to shorter operating times.

The present disclosure is explained in more detail below with reference to the figures. Where appropriate, elements with the same function carry the same reference signs. The present disclosure is not restricted to the exemplary embodiments illustrated in the figures—also not in relation to functional features. The above description and the following description of the figures contain numerous features which may be partially combined in the dependent subclaims. These features, and all other features disclosed above and in the following description of the figures, may also be considered individually by the person skilled in the art and combined into suitable further combinations. In particular, all cited features may be achieved individually and in arbitrary suitable combination with the medical tool tip according to the first aspect of the present disclosure, the actuation device according to the second aspect of the present disclosure, the surgical instrument according to the third aspect of the present disclosure, and the method according to the fourth aspect of the present disclosure.

### BRIEF DESCRIPTION OF THE DRAWINGS

The drawings show, at least partially schematically:

FIG. 1 an example of a surgical instrument;

FIG. 2 components of the surgical instrument from FIG. 1;

FIG. 3 an example of a medical tool tip in a side view;

FIG. 4 the medical tool tip from FIG. 3 in exploded form;

FIG. 5 the medical tool tip from FIG. 3 in a top view;

FIG. 6 an example of a distal end of an actuation device in a sectional view;

FIG. 7 the distal end of the actuation device from FIG. 6 in a three-dimensional illustration; and

FIG. 8 an example of a method for attaching a medical tool tip to an actuation device.

### DETAILED DESCRIPTION

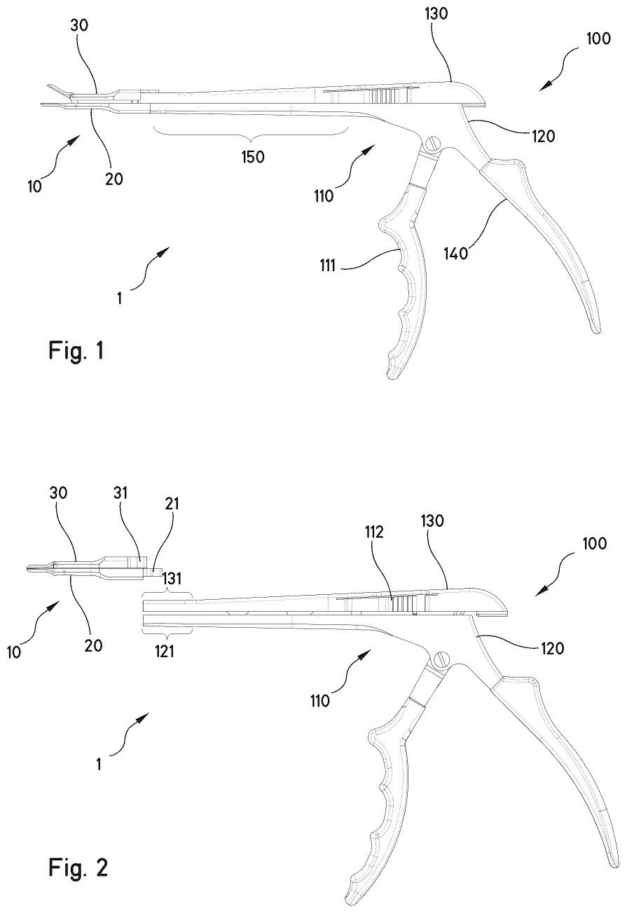

FIG. 1 shows an example of a surgical instrument 1 with a medical tool tip 10 and an actuation device 100. The tool tip 10 has a first tip part 20 and a second tip part 30 which is attached to the first tip part 20 so as to be at least partially longitudinally displaceable. The actuation device 100 has a base body 120 and a slide 130 which is attached to the base body 120 so as to be at least partially longitudinally displaceable. The longitudinal displacement of the slide 130 relative to the base body 120 may be triggered for example by means of an actuation mechanism 110.

At a proximal end, i.e. the end nearest a user during use of the instrument 1, the actuation device 100 preferably has a handle 140. It is suitable if the handle 140 is formed by the base body 120. Another part of the base body 120, together with a corresponding portion of the slide 130, forms a shank 150.

The tool tip 10 is arranged at a distal end, i.e. an end remote from the user during use of the surgical instrument 1, of the actuation device 100, in particular the shank 150. The tool tip 10 is an exchangeable tool tip (see FIG. 2) and in the present illustration is connected to the actuation device 100 ready for operation. Suitably, for this the first tip part 20 is attached to the base body 120 and the second tip part 30 is attached to the slide 130. For example, because of a shorter design of the second tip part 30 relative to the first tip part 20, the slide 130 here protrudes over the base body 120 at the distal end of the actuation device 100.

When the actuation mechanism 110 is triggered, in particular by pulling of a trigger lever 111, the slide 130 moves relative to the base body 120 towards the distal end. Because of the connection of the slide 130 to the second tip part 30, the tool tip 10 may thus be actuated.

Purely as an example, the tool tip 10 shown in FIG. 1 comprises so-called spoon forceps or grasping forceps. The surgical instrument 1 is however also conceivable with another tool tip, e.g. a punch, a disc attachment, a needle holder, a clamp or shears.

FIG. 2 shows components of the surgical instrument 1 from FIG. 1, namely in particular the actuation device 100 and the tool tip 10 detached therefrom. A first attachment portion 21 of the first tip part 20 and a second attachment portion 31 of the second tip part 30 are therefore visible. The slide 130 is in an attachment position relative to the base body 120, in which position in particular the first tip part 20 can be attached to (or detached from) the base body 120.

The attachment portions 21, 31 suitably serve for attachment of the first and second tip parts 20, 30 to the base body 120 or slide 130. For attachment to the base body 120, the first attachment portion 21 can here preferably be inserted in a base body attachment portion 121 at the distal end of the base body. In other words, the base body 120 in the base body attachment portion 121 is suitably configured to receive the first attachment portion 21, in particular by form fit.

It is suitable if the base body attachment portion 121 is only accessible for the first attachment portion 21 when the slide 130 is in the attachment position. To this extent, it is preferred if the slide 130 secures the first attachment portion 21 in the base body attachment portion 121 in at least one operating position. Such an operating position suitably exists if the slide 130—in the state of the surgical instrument 1 ready for operation as shown in FIG. 1—protrudes at the distal end beyond the base body 120, in particular the base body attachment portion 121.

For attachment of the second tip part 30 to the slide 130, the second attachment portion 31 can preferably be inserted in a slide attachment portion 131 at the distal end of the slide 130. In other words, the slide 130 in the slide attachment portion 131 is suitably configured to receive the second attachment portion 31, in particular by form fit. The slide attachment portion 131 may be configured for pushing onto, in particular engaging on, the second attachment portion 31. A connection between the slide 130 and the second tip part 30 can here preferably easily be created by the longitudinal displacement of the slide 130 relative to the base body 120, i.e. by actuation of the actuation mechanism 110.

Suitably, the slide 130 is raised from the base body 120 in the attachment position, as shown in FIG. 2. This can facilitate not only the detachment and attachment of the first tip part 20 from or to the base body 120, but also cleaning or sterilization of the actuation device 100. Therefore the attachment position may also be described as a cleaning or sterilization position. The actuation device 100 can be brought out of the operationally ready state shown in FIG. 1 into the attachment position, for example in that a locking of the actuating mechanism 110 is released and the slide 130 is manually drawn towards the proximal end relative to the base body 120. To release the locking, suitably a corresponding actuation element 112 is provided on the slide 130.

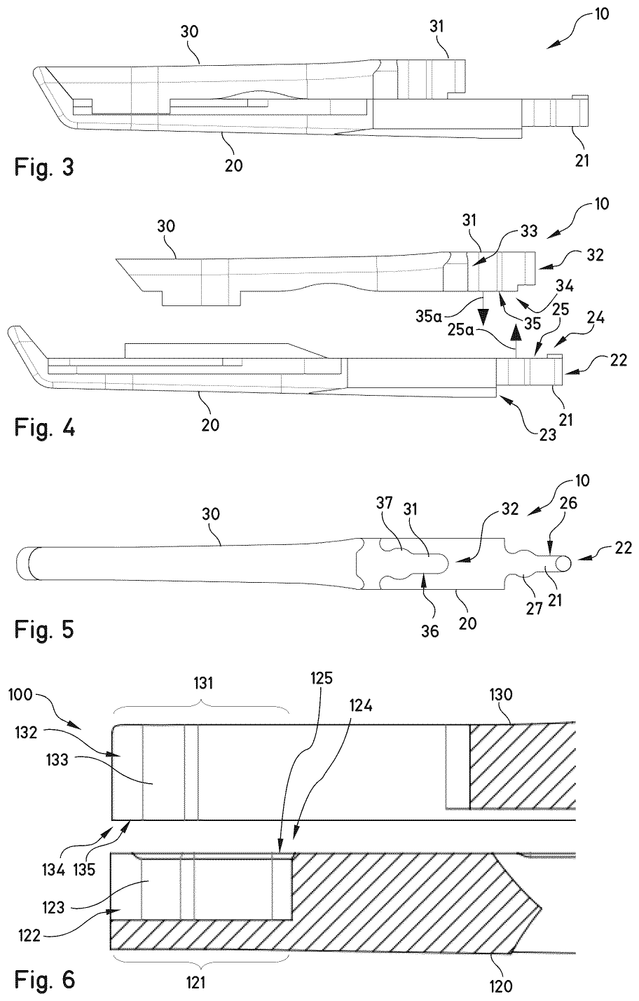

FIG. 3 shows an example of a medical tool tip 10 in a side view. The tool tip 10 has a first tip part 20 and a second tip part 30 which is attached longitudinally displaceably on the first tip part 20. The first tip part 20 has a first attachment portion 21, and the second tip part 30 has a second attachment portion 31, which are configured respectively for attachment to a base body or slide of an actuation device (see FIGS. 1 and 2).

Suitably, the first attachment portion 21 and the second attachment portion 31 are configured, in particular arranged, such that firstly, the first attachment portion 21 can be attached to the base body and then the second attachment portion 31 can be attached to the slide. In other words, the first and second attachment portions 21, 31 may be configured, in particular arranged, such that a two-stage attachment of the tool tip 10 to the actuation device is possible. For example, firstly the first attachment portion 21 may be inserted in a base body attachment portion of the base body, whereupon a slide attachment portion of the slide can be pushed onto the second attachment portion 31 by longitudinal displacement relative to the base body. In particular, by attachment of the first attachment portion 21 to the base body, the second attachment portion 31 is positioned and/or held so as to allow or at least simplify the creation of a connection between the second attachment portion 31 and the slide.

For the two-stage attachment to the actuation device, the attachment portions 21, 31 may for example be spaced from one another or be able to be spaced from one another by longitudinal displacement, in particular in a longitudinal direction of the tool tip 10. Such an arrangement can easily be achieved if the second tip part 30 is formed shorter than the first tip part 20.

FIG. 4 shows the tool tip 10 from FIG. 3 in exploded form. Here, a possible design of the first and second attachment portions 21, 31 is shown. The first attachment portion 21 and the second attachment portion 31 may here have a respective first or second protrusion 22, 32 which, running longitudinally, adjoins a proximal end 23, 33 of the respective tip part 20, 30. In particular, the first and second attachment portions 21, 31 may be defined by the respective protrusion 22, 32. The protrusions 22, 32 are suitably formed such that, in particular, they can be received by form fit, firmly seated, by the base body in the base body attachment portion or by the slide in the slide attachment portion.

In order to allow a smooth actuation of the tool tip 10, it is suitable if the first tip part 20, in particular the first attachment portion 21, has a top side 24 with a flat top side face 25. The top side 24 suitably faces an underside 34 of the second tip part 30, in particular of the second attachment portion 31. The underside 34 suitably has a flat underside face 35.

It is preferred that the top side face 25 and underside face 35 suitably run parallel with one another. FIG. 4 shows the corresponding surface normals 25a, 35a of the top side face 25 and underside face 35 respectively.

FIG. 5 shows the tool tip 10 from FIG. 3 in a top view. This illustrates a possible form of the first tip part 20 in the first attachment portion 21, in particular the first protrusion 22, and the second tip part 30 in the second attachment portion 31, in particular the second protrusion 32.

The first and second protrusions 22, 32 suitably have side walls 26, 36. These side walls 26, 36 may run perpendicularly to the top side face and underside face shown in FIG. 4, i.e. perpendicularly to the drawing plane in FIG. 5. Preferably, the side walls 26, 36 are formed unevenly for reliable, in particular form-fit attachment to the actuation device.

For example, the first and second attachment portions 21, 31 may have a respective first and second bulge 27, 37. These bulges 27, 37 suitably run transversely to the surface normals of the top side face and underside face, shown in FIG. 4. In other words, the protrusions 22, 32 suitably have a lateral thickening, i.e. a thickening arranged in the region of the side walls 26, 36.

These bulges 27, 37 preferably each have a convex curvature. The first and second attachment portions 21, 31 may thus be formed such that the base body or slide can undercut the respective first or second attachment portion 21, 31. An undesirable detachment of the tool tip 10 from the distal end of the actuation device in the longitudinal direction can thereby be prevented.

As FIG. 5 clearly shows, in a possible embodiment, the first attachment portion 21 and the second attachment portion 31 have at least partially and at least substantially the same contour, in particular the same outer contour. In particular, the top side face 25 marked in FIG. 4, and the underside face 35 also marked there, may be at least partially similarly formed. To this extent, it is suitable if the side walls 26, 36 are formed similarly uneven, i.e. for example the second bulge 37 is formed at least substantially the same as the first bulge 27.

Here, it is not necessary for the contours of the attachment portions 21, 31 to be identical, i.e. to match one another without scaling. Rather, in particular the first attachment portion 21 may be dimensioned smaller, in particular formed narrower, than the second attachment portion 31. It is however preferred that the contours are similar, i.e. at least partially match one another with corresponding slight scaling.

In the case of a two-stage attachment of the tool tip 10 to the actuation device, such a matching of the first attachment portion 21 and the second attachment portion 31 allows the first attachment portion 21 to be secured in the base body attachment portion, and the slide attachment portion to be pushed onto the second attachment portion 31, by a single actuation of the actuation device. A smaller dimensioning of the first attachment portion 21 can ensure that this can easily be conducted through the slide attachment portion. This is evident in connection with FIG. 6.

FIG. 6 shows an example of a distal end of an actuation device 100 in a sectional view. The actuation device 100 has a base body 120 and a slide 130 which is longitudinally displaceably attached to the base body 120. The slide 130 is here in the attachment position, already shown in FIG. 2, in which it is raised from the base body 120 running parallel thereto. Thus FIG. 6 shows a base body top side 124 with a base body sliding face 125 of the base body 120, which faces a slide underside 134 of the slide 130 with a slide sliding face 135. The slide sliding face 135 is suitably configured to slide along the base body sliding face 125 on actuation of the actuation device 100.

The base body 120 has a base body attachment portion 121 for attachment of a first tip part of a medical tool tip (see FIGS. 3, 4 and 5). The base body attachment portion 121 may in particular be configured to receive a first attachment portion of the first tip part. For this, it is suitable for the base body attachment portion 121 to be configured, in particular formed, correspondingly to the first attachment portion. In particular, the base body attachment portion 121 may have a base body recess 122, into which a first protrusion of the first tip part (see FIGS. 4 and 5) can be inserted with form fit, i.e. which is formed correspondingly to the first protrusion.

The slide 130 has a slide attachment portion 131 for attachment of a second tip part of the tool tip (see FIGS. 3, 4 and 5). The slide attachment portion 131 may in particular be configured to receive a second attachment portion of the second tip part. For this, it is suitable for the slide attachment portion 131 to be configured, in particular formed, correspondingly to the second attachment portion. In particular, the slide attachment portion 131 may have a slide recess 132, into which a second protrusion of the second tip part (see FIGS. 4 and 5) can be inserted with form fit, i.e. which is formed correspondingly to the second protrusion.

For example, both the base body attachment portion 121 and also the slide attachment portion 131 may have an indentation 123, 133. These indentations 123, 133 may be configured to receive corresponding bulges of the first and second attachment portions (see FIG. 5).

As FIG. 6 clearly shows, the base body attachment portion 121 and the slide attachment portion 131 have at least partially substantially the same contour, in particular the same inner contour. For example, the base body sliding face 125 and the slide sliding face 135 may have at least partially the same contour.

In particular, it is suitable that—as shown in FIG. 6—the contours of the base body attachment portion 121 and slide attachment portion 131 at least partially substantially align with one another when the slide 130 is in the attachment position. In particular, the base body sliding face 125 and the slide sliding face 135 may at least partially align with one another. Thus a first attachment portion formed correspondingly to the base body attachment portion 121 can easily be inserted into the base body attachment portion 121 through the slide attachment portion 131.

If the slide 130 is longitudinally displaced relative to the base body 120 out of the attachment position, the slide 130 can secure the first attachment portion which is received by the base body attachment portion 121. In other words, in such an operating position, the slide 130 may prevent the first attachment portion from being (undesirably) extracted from the base body attachment portion 121 through the slide attachment portion 131. This may be achieved for example if a part of the slide 130 at least partially overlaps the indentation 123 in the base body attachment portion 121.

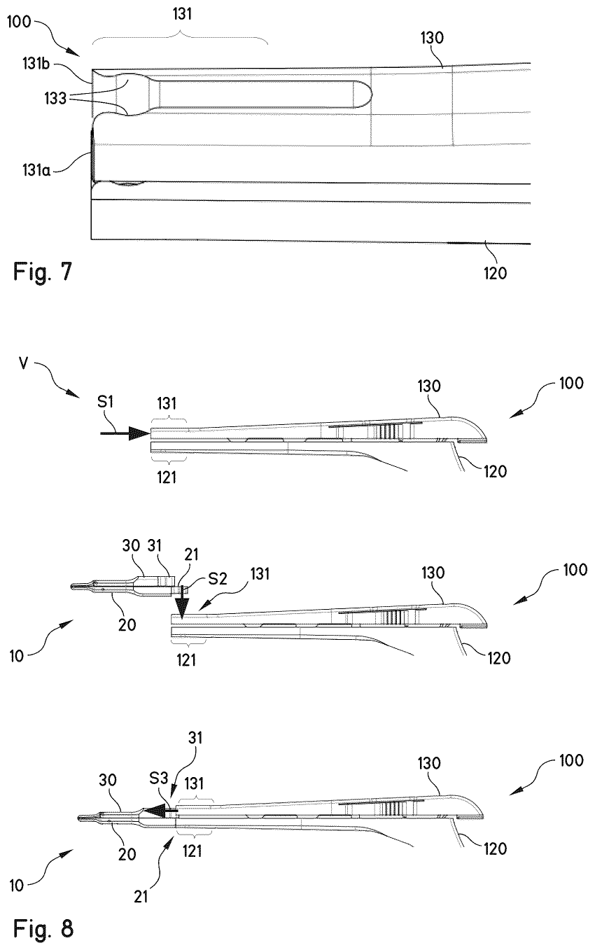

The attachment of the second tip part to the slide 130 is explained in connection with FIG. 7.

FIG. 7 shows the distal end of the actuation device 100 from FIG. 6 in a three-dimensional illustration. It is clearly evident that the slide 130 in the slide attachment portion 131 is suitably forked. The slide 130 may for example have two fork tines 131a, 131b. The fork tines 131a, 131b are preferably blade-like, i.e. thin relative to a longitudinal extent. This may achieve a flexibility of the slide attachment portion 131 which allows the slide attachment portion 131 to be pushed onto the second attachment portion, which is suitably formed correspondingly to the slide attachment portion 131, by a longitudinal displacement.

For example, on a longitudinal movement towards the second tip part, the fork tines 131a, 131b may be pressed apart or bent laterally by the bulge in the second attachment portion (see FIG. 5). When the bulge finally reaches the indentations 133, the slide attachment portion 131 may engage on the second tip part by undercutting the bulge.

FIG. 8 shows an example of a method V for attaching a medical tool tip 10 to an actuation device 100.

In a method step S1, a slide 130 of the actuation device 100 is brought into an attachment position by longitudinal displacement relative to a base body 120 of the actuation device 100. Then the slide 130 can particular be longitudinally displaced such that a contour of a slide attachment portion 131 aligns with a contour of a base body attachment portion 121. For example, a slide recess of the slide attachment portion 131 may be oriented in alignment with a base body recess of the base body attachment portion 121.

In a further method step S2, a first attachment portion 21 of a first tip part 20 of the tool tip 10 is attached in the base body attachment portion 121 the base body 120. In particular, the first attachment portion 21 may then be inserted from above the actuation device 100, i.e. from a top side of the slide 130 facing away from the base body 120, as indicated by the arrow, into the base body attachment portion 121, in particular the base body recess. The first attachment portion 21 is here suitably conducted through the slide attachment portion 131.

In a further method step S3, a second attachment portion 31 of a second tip 30 of the tool tip 10 is attached by longitudinal displacement of the slide 130 relative to the base body 120 out of the attachment position. Suitably, the first attachment portion 21 is here simultaneously secured in the base body attachment portion 121, for example in that the slide 130 at least partially overlaps the base body attachment portion 121 which is open at the top in an actuation position.

Suitably, the slide attachment portion 131 pushes onto the second attachment portion 31 in a longitudinal direction, as indicated by the arrow, until the slide 130 engages on the second attachment portion 31. The second attachment portion 31 is here preferably laterally surrounded.

Accordingly, it is possible, by simple actuation of the actuation device 100, to create a connection between the slide 130 and the second tip part 30, and at the same time fix the first tip part 20 to the base body 120. This may mean a significant increase in user-friendliness and allow safe operation of the surgical instrument assembled in this way.

### LIST OF REFERENCE DESIGNATIONS

1 Surgical instrument
10 Medical tool tip
20 First tip part
21 First attachment portion
22 First protrusion
23 Proximal end of first tip part
24 Top side
25 Top side face
25a Surface normal of top side face
26 Side wall
27 First bulge
30 Second tip part
31 Second attachment portion
32 Second protrusion
33 Proximal end of second tip part
34 Underside
35 Underside face
35a Surface normal of underside face
36 Side wall
37 Second bulge
100 Actuation device
110 Actuation mechanism
111 Trigger lever
112 Actuation element
120 Base body
121 Base body attachment portion
122 Base body recess
123 Indentation
124 Base body top side
125 Base body sliding face
130 Slide
131 Slide attachment portion
131a Fork tines
131b Fork tines
132 Slide recess
133 Indentation
134 Slide underside
135 Slide sliding face
140 Handle
150 Shank
V Method
S1-S3 Method steps

1. A medical tool tip (10) for a surgical instrument (1), the medical tool tip comprising:
a first tip part (20) having a first attachment portion (21) for toolless attachment of the first tip part (20) to a base body (120) of an actuation device (100), and
a second tip part (30) having a second attachment portion (31) for toolless attachment of the second tip part (30) to a slide (130) of the actuation device (100), wherein the second tip part (30) is at least partially longitudinally displaceably attached to the first tip part (20),
wherein the first and second attachment portions (21, 31) are configured such that the second tip part (30) is configured to be attached to the slide (130) only when the first tip part (20) has already been attached to the base body (120).

2. The tool tip (10) as claimed in claim 1, wherein the first attachment portion (21) and the second attachment portion (31) have at least partially a same contour.

3. The tool tip (10) as claimed in claim 1, wherein:
the first tip part (20) has a top side (24) which faces the second tip part (30) and has a flat first contact face,
the second tip part (30) has an underside (34) which faces the first tip part (20) and has a flat second contact face, via which the second tip part (30) contacts the first contact face in at least one operating position, and
the first contact face and the second contact face are at least partially similarly formed.

4. The tool tip (10) as claimed in claim 1, wherein:
the first attachment portion (21) has a first protrusion (22) which, running longitudinally, adjoins a proximal end (23) of the first tip part (20) and has a top side (24) with a flat top side face (25),
the second attachment portion (31) has a second protrusion (32) which, running longitudinally, adjoins a proximal end (33) of the second tip part (30) and has an underside (34) with a flat underside face (35) which is oriented substantially parallel to the top side face (25), and
the first and second protrusions (22, 32) have respective side walls (26, 36) which run substantially perpendicularly to the top side face (25) and underside face (35) and are formed similarly uneven.

5. The tool tip (10) of claim 1, wherein:
the first tip part (20) in the first attachment portion (21) has a top side (24) with a top side face (25) and a first bulge (27), which runs transversely to a surface normal (25a) of the top side face (25), and
the second tip part (30) in the second attachment portion (31) has an underside (34) with an underside face (35) facing the top side face (25) and a second bulge (37), which runs transversely to a surface normal (35a) of the underside face (35) and is formed similarly to the first bulge (27).

6. The tool tip (10) as claimed in claim 5, wherein the first tip part (20) and the second tip part (30) each have a convex curvature in the region of the respective first or second bulge (27, 37).

7. An actuation device (100) for a surgical instrument (1), the actuation device comprising:
a base body (120) having a base body attachment portion (121) for toolless attachment of a first tip part (20) of a medical tool tip (10) to the base body (120), and
a slide (130) having a slide attachment portion (131) for toolless attachment of a second tip part (30) of the tool tip (10), which is longitudinally displaceably attached to the first tip part (20), to the slide (130), wherein the slide (130) is at least partially longitudinally displaceably attached on the base body (120),
wherein, for toolless attachment of the tool tip (10) to the actuation device (100), the slide (130);
is configured to be brought into an attachment position by at least partial longitudinal displacement relative to the base body (120), in which position the first tip part (20) is configured to be attached to the base body attachment portion (121) of the base body (120), and
is configured such that, when the first tip part (20) is attached to the base body (120), the second tip part (30) is configured to be connected to the slide (130) in the slide attachment portion (131) by at least partial longitudinal displacement of the slide (130) out of the attachment position.

8. The actuation device (100) as claimed in claim 7, wherein a contour of the base body attachment portion (121) and a contour of the slide attachment portion (131) at least partially substantially align with one another when the slide (130) is in the attachment position.

9. The actuation device (100) as claimed in claim 7, wherein:
the base body (120) has a base body top side (124) facing the slide (130) with a flat base body sliding face (125),
the slide (130) has a slide underside (134) facing the base body (120) with a flat slide sliding face (135), via which the slide (130) at least partially slides along the base body sliding face (125) on a longitudinal displacement relative to the base body (120), and
the base body sliding face (125) and the slide sliding face (135) at least partially substantially align with one another when the slide (130) is in the attachment position.

10. The actuation device (100) as claimed in claim 7, wherein the base body attachment portion (121) has a base body recess (122), and the slide attachment portion (131) has a slide recess (132), which at least partially substantially align with one another in the attachment position.

11. The actuation device (100) as claimed in claim 7, wherein the slide attachment portion (131) is configured to secure the first tip part (20) attached to the base body attachment portion (121).

12. The actuation device (100) as claimed in claim 7, wherein the slide (130) is configured to release the first tip part (20) attached to the base body attachment portion (121) in the attachment position.

13. The actuation device (100) as claimed in claim 7, wherein the slide (130) in the slide attachment portion (131) is formed as a fork for at least partially surrounding the second tip part (30).

14. The actuation device (100) as claimed in claim 7, wherein the slide attachment portion (131) has two fork tines (131a, 131b) which are designed to be deformable for attaching the second tip part (30) by longitudinal displacement of the slide (130).

15. The actuation device (100) as claimed in claim 7, wherein the slide attachment portion (131) is configured to engage on the second tip part (30) for attachment.

16. A surgical instrument (1) having an actuation device (100) as claimed in claim 7, and a medical tool tip (10) comprising:
a first tip part (20) having a first attachment portion (21) for toolless attachment of the first tip part (20) to a base body (120) of an actuation device (100), and
a second tip part (30) having a second attachment portion (31) for toolless attachment of the second tip part (30) to a slide (130) of the actuation device (100), wherein the second tip part (30) is at least partially longitudinally displaceably attached to the first tip part (20),
wherein the first and second attachment portions (21, 31) are configured such that the second tip part (30) is configured to be attached to the slide (130) only when the first tip part (20) has already been attached to the base body (120).

17. A method (V) for attaching a medical tool tip (10) with a first tip part (20) and a second tip part (30), which is attached longitudinally displaceably on the first tip part (20), to an actuation device (100) with a base body (120) and a slide (130) which is longitudinally displaceably attached on the base body (120), the method comprising:
(S1) bringing the slide (130) into an attachment position by longitudinal displacement relative to the base body (120);
(S2) toollessly attaching the first tip part (20) using a first attachment portion (21) on the base body (120) in a base body attachment portion (121);
(S3) toollessly attaching the second tip part (30) using a second attachment portion (31) on the slide (130) in a slide attachment portion (131), by longitudinal displacement of the slide (130) relative to the base body (120) out of the attachment position.
